(19) United States
(12) Patent Application Publication
(10) Pub. No.: US 2011/0229414 A1
(43) Pub. Date: Sep. 22, 2011

(54) Contrast Agents For Detecting Protease Activity By Means Of Hyperpolarization And For Stratifying Patients

(75) Inventors: Christian Geppert, Erlangen (DE); Arne Hengerer, Mohrendorf (DE); Sven Meyburg, Erlangen (DE); Sebastian Schmidt, Weisendorf (DE); Susanne Schmolke, Erlangen (DE); Ralph Markus Wirtz, Koln (DE)

(73) Assignees: SIEMENS AKTIENGESELLSCHAFT, Munich (DE); SIEMENS HEALTHCARE DIAGNOSTICS PRODUCTS GMBH, Marburg (DE)

(21) Appl. No.: 13/013,127

(22) Filed: Jan. 25, 2011

(30) Foreign Application Priority Data
Jan. 27, 2010 (DE) 10 2010 005 880.7

(51) Int. Cl. A61K 49/08; C07K 2/00; A61K 49/00; C07K 7/06; A61P 43/00

(52) U.S. Cl. 424/9.3; 530/300; 424/9.1; 530/329

(57) ABSTRACT

A contrast agent is disclosed for imaging methods. In at least one embodiment the contrast agent includes a construct including i) at least two copies of a substrate for at least one tumor-specific protease, and ii) at least one linker having at least one recognition site for at least one tumor-specific protease, wherein a hyperpolarization site is located at the N and/or C terminus of the substrate, and wherein the linker is configured such that the hydrophobic ends of the substrate interact and form a central core by means of noncovalent interactions with the lipophilic residues, and the contrast agent together with a parahydrogen metal template for use in an imaging method for diagnosing a tumor. At least one embodiment of the invention further relates to a method for imaging a tumor tissue, wherein the above-described contrast agent accumulated in a tumor tissue is imaged using a suitable imaging method after hyperpolarization by way of contact with a parahydrogen metal template, and also to a method for imaging a tumor tissue in a patient, wherein a) a contrast agent as described above is administered to a patient, b) a parahydrogen metal template is administered to the patient, and c) the presence of a tumor is depicted using an imaging method.

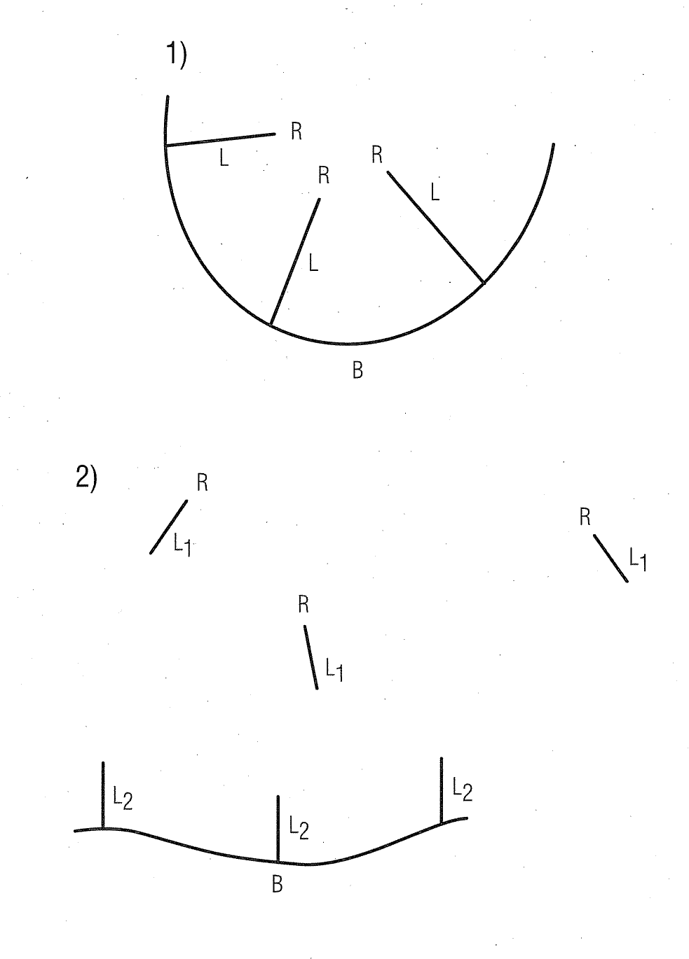

### PRIORITY STATEMENT

The present application hereby claims priority under 35 U.S.C. §119 on German patent application number DE 10 2010 005 880.7 filed Jan. 27, 2010, the entire contents of which are hereby incorporated herein by reference.

### FIELD

At least one embodiment of the invention generally relates to contrast agents for imaging methods which comprise a macromolecular construct which comprises both an activation site for a protease and a hyperpolarization site. At least one embodiment of the invention further generally relates to the use of this contrast agent together with a hyperpolarization agent for use in an imaging method for diagnosing a tumor and also to a method for imaging a tumor tissue using this contrast agent after hyperpolarization. In addition, at least one embodiment of the invention relates to a method for imaging a tumor tissue in a patient, where both the contrast agent and a parahydrogen metal template are administered to the patient. At least one embodiment of the invention further generally relates to a method for stratifying patients by way of the combined use of in vitro and in vivo diagnostic methods.

### BACKGROUND

Molecular biomarkers which indicate the occurrence of a tumor at an early stage or allow the course of a tumor to be predicted are increasingly important in modern medicine. The use of suitable biomarkers makes it possible to diagnose tumors at an early stage and with greater reliability. An improved prognosis owing to the determination of molecular biomarkers makes it possible to develop an optimal treatment strategy for cancer patients. Molecular biomarkers are also used in the diagnosis of tumors or cancers by means of imaging methods. For this purpose, use is often made of contrast agents which bind to defined biomarkers in the tumor tissue in a targeted manner and thus increase the validity of an examination using an imaging method.

MR contrast agents which bind to certain molecular biomarkers in the target tissue and accumulate locally as a result have proven to be not so suitable for most diagnostic applications. Since most molecular biomarkers are present at a low concentration, there is no local concentration of MR contrast agents in the micromolar range sufficient for detection by MR.

However, image-based molecular diagnostics are unavoidable for spatially resolved detection of biomarkers in the submicromolar range (preferably, nanomolar sensitivity).

Furthermore, the specificity of an individual tumor marker is often limited, since expression thereof can also be found in healthy tissues or other pathologically altered tissue regions.

A large variety of approaches have been developed for the detection of molecular biomarkers by means of MR (amplification mechanisms). Thus, enzymatically activatable contrast agents have been developed which are applied in an inert state and are converted to an active (i.e., detectable by means of MR) state by enzymes (e.g., proteases) expressed selectively in the target tissue. A further approach for increasing sensitivity is based on hyperpolarization of the contrast agent (K GOLMAN et al., The British Journal of Radiology, 76 (2003), pages 118-127).

Of particular importance with regard to the diagnosis and prognosis of tumors is the detection of the interaction of tumor cells with the surrounding tissue. For example, the activation of surrounding healthy connective tissue cells or the recruitment of immune cells or bone marrow stem cells is needed so that a tumor cell that has migrated to a distant region of the body can actively divide again, develop into a macrometastasis, and eventually colonize further regions of the body.

Tissue examinations have revealed that increased and tumor cell-specific mRNA expression levels of proteases and, in particular, of the matrix metalloproteinases MMP-1, MMP-2, MMP-7, MMP-9, and MMP-12 are associated with a poor prognosis for cancer patients (Gentner B et al., Anticancer Res. 2009 January; 29(1): 67-74). However, expression of the MMP mRNA is only detectable in a comparatively small portion of the primary tumors. Also, translation of the MMP mRNA initially leads only to the synthesis of inactive precursors of the enzymatically active version of the protein (=proenzyme). Furthermore, the activity of the MMP enzymes is also influenced by the simultaneous presence of specific inhibitors. Accordingly, the detection of MMP mRNA or protein is diagnostically imprecise, despite the described prognostic significance in specific cancer patient subgroups.

### SUMMARY

There is thus a need in the prior art to expand and to improve the diagnostic potential of existing molecular biomarkers. Thus, at least one embodiment of the invention is intended to provide an improved contrast agent for imaging methods. In addition, at least one embodiment of the invention is intended to provide an improved method for imaging tumor tissues. The contrast agent according to at least one embodiment of the invention is intended to be simple to use with very little secondary effects. It is further intended to be easy to use, to have very little secondary effects for the patient, and also to allow a reliable diagnosis of a tumor.

At least one embodiment of the invention relates to an MR contrast agent strategy which enables two amplification mechanisms to be combined: enzymatic activation and hyperpolarization. Protease-initiated cleavage of linkers makes it possible to release active ingredients, containing peptide linkers, in the body in a targeted manner (as discussed in German patent application number DE 10 2007 042 107 A1, the entire contents of which are hereby incorporated herein by reference). Peptide linkers can likewise be used for the targeted release of contrast agents (as discussed in German patent application number DE 10 2007 028 090 A1, the entire contents of which are hereby incorporated herein by reference). It was found that, surprisingly, combining these two amplification mechanisms increases the specificity of an examination using an imaging method. Signal amplification mechanisms based on enzymatic activation and hyperpolarization enable detection of prognostic biomarkers without the need for a biopsy or reduce the need for a rebiopsy.

At least one embodiment of the invention relates to a contrast agent for imaging methods, comprising a construct of i) at least two copies of a substrate for at least one tumor-specific protease and ii) at least one linker having at least one recognition site for at least one tumor-specific protease, wherein a hyperpolarization site is located at the N and/or C terminus of the substrate, and wherein the linker is configured such that the hydrophobic ends of the substrate interact and form a central core by means of noncovalent interactions with the lipophilic residues. At least one embodiment of the invention further relates to this contrast agent together with a parahydrogen metal template for use in an imaging method for diagnosing a tumor.

In addition, at least one embodiment of the invention relates to a method for imaging a tumor tissue, wherein the contrast agent accumulated in a tumor tissue is imaged using a suitable imaging method after hyperpolarization by way of contact with a parahydrogen metal template. There is further described a method for imaging a tumor tissue in a patient, wherein a) the above contrast agent is administered to a patient, b) a parahydrogen metal template is administered to the patient, and c) the presence of a tumor is depicted using an imaging method.

Preferably, the selection of the contrast agent is supported by upstream in vitro diagnostic methods or by using the results obtained with such methods. Thus, by way of example, a tumor sample taken from a patient is tested for the presence of one or more tumor-specific proteases. If one or more tumor-specific proteases are present, the contrast agent which contains one or more recognition sites for this/these tumor-specific protease(s) can then be used in a targeted manner. This increases the specificity of the imaging method, i.e., the level of false-positive or false-negative results can be minimized in this way. As a result of the patient being administered, in a targeted manner, the contrast agent according to at least one embodiment of the invention and appropriate for his/her disease, patients are not subjected unnecessarily to contrast agent doses which may be a strain on the metabolism of the patient or may have an allergenic potential. Particular preference is given to testing for the presence of the following tumor-specific proteases:

Presence of at least one tumor-specific protease selected from MMP-7 and/dr PSA

Presence of MMP-9 and MMP-7

Presence of PSA and MMP-9 and MMP-7

Presence of PSA and MMP-7

Presence of PSA and MMP-9

A preferred embodiment of the invention thus relates to a method for imaging a tumor tissue, wherein upstream in vitro diagnostic methods for determining the presence of one or more tumor-specific proteases are used in the selection of a suitable contrast agent according to at least one embodiment of the invention and, subsequently, the contrast agent accumulated in a tumor tissue is imaged with a suitable imaging method after hyperpolarization by means of contact with a parahydrogen metal template. This embodiment of the invention allows the aggressiveness or the metastatic potential of a tumor to be assessed at an early stage.

The activation of the proteases is carried out in a highly controlled manner, in a specific sequence, and determines in the case of neoplastic diseases the interplay between tumor cells and nontumor cells. This interaction takes place in particular at invasive fronts of tumors and during penetration of the basal lamina, this being a characteristic for discriminating benign and malignant tumors. The localizability of the MMP activity in conjunction with further imaging information and the probability, deduced from the tissue examination, of the presence of this activity increase synergistically the specificity of the detection. The use of the contrast agent disclosed in at least one embodiment of this invention and having two different protease sites reveals the simultaneous presence of two protease activities at a molecular spatial resolution. Both proteases have to be active on a single molecule in order to produce the imaging signal.

The simultaneous measurement of cancer-associated activities which are produced either specifically by the tumor cells (MMP-7) or by recruited macrophages or by way of fibroblasts activated by tumor cells (MMP-9) increases the specificity of the detection, since different markers which have limited tumor specificity must be present and active at the same time.

A combination of in vitro methods and the result of in vivo diagnostic methods according to one embodiment of the invention enables individualization of diagnostic and therapeutic measures. A better estimation of the malignancy of the tumor and of the effectiveness of therapies is achieved. This prevents overtreatment and counteracts undertreatment at an earlier stage. By selecting the most suitable patients for the imaging methods with the aid of the results of the upstream in vitro diagnostics, the advantages of the additional imaging measures become clear and implementing the modified clinical workflow is facilitated while saving resources at the same time. Selecting patients for imaging prevents their unnecessary subjection to additional examinations.

Ralph W. Adams published a study entitled “Reversible Interactions with para-Hydrogen Enhance NMR Sensitivity by Polarization Transfer” (27 Mar. 2009, vol. 323 SCIENCE), the entire contents of which are hereby incorporated herein by reference. The publication describes a method for the direct, parahydrogen-mediated hyperpolarization of a contrast agent without the need for hydrogenation. The hyperpolarization is achieved by way of a temporary association of a substrate with parahydrogen in the presence of a transition metal center at a low field intensity.

In the cited publication, the substrates used were pyridine and [Ir(COD)(PCy3)(MeCN)][BF4] (Cy=cyclohexyl, and COD=cyclooctadiene) and an iridium dihydride complex [Ir(H)2(PCy3)(pyridine)3] was produced. The reaction conditions are very simple. It is sufficient to shake the reactants at a low field intensity. According to this principle, hyperpolarization can be transferred to different substrates by means of different metals.

According to at least one embodiment of the invention, the reversible interaction of parahydrogen with the substrate is used to activate an inert contrast agent in vivo. For this purpose, use is made of a contrast agent which is a macromolecular construct comprising at least two copies of a substrate for at least one tumor-specific protease and at least one linker having at least one recognition site for at least one tumor-specific protease. A hyperpolarization site is located at the N and/or C terminus of the substrate. The linker is configured such that the hydrophobic ends of the substrate interact and form a central core by way of noncovalent interactions with the lipophilic residues.

The substrate can be any substrate for a tumor-specific protease. Preferably, the substrate is a heterocycle (heteroaromatic compound), for example the aromatic amino acids tryptophan or histidine. In the construct according to the invention, at least two copies of this substrate are present. However, the substrate is preferably present at a higher copy number, for example in the range from 10 to 500 copies, preferably from 50 to 400 copies, more preferably from 100 to 300 copies.

The copies of the substrate are connected by a linker having protease recognition sites such that the hydrophobic ends interact and form a central core by means of noncovalent interactions with the lipophilic residues. This core is protected sterically and therefore it is not accessible to a metal complex.

Furthermore, multiple linkers can be bound to one physiologically compatible backbone structure. The basic structure of the linker bound to a backbone structure can be found in the accompanying figure. It can be seen in this figure that various linkers are bound to one flexible backbone structure, forming a central core. The backbone structure consists of a physiologically compatible polymer, for example dextran, starch, polylysine. Corresponding structures are known in the field of pharmacy. The number of bound linkers depends on the length of the linker. Preferably, there is one linker per monomeric unit of the backbone structure.

If necessary, steric shielding of the central core is mediated by further space-filling residues coupled to the linker. Such residues are known to a person skilled in the art. An example is polyethylene glycol.

The linker as such is preferably a peptide, for example: Ala-Gly-Cys(Me)-His-Ala-Lys(Nma)-NH₂ for the proteases MMP-1, MMP-3, MMP-7, MMP-8, MMP-9, MMP-11, MMP-12, and MMP-13.

Located on this linker is a protease recognition site for a tumor-specific protease.

### BRIEF DESCRIPTION OF THE DRAWINGS

Further advantages, features and properties of the present invention are explained below in more detail with the aid of example embodiments and with reference to the accompanying drawings, in which:

The figure shows the construct according to an embodiment of the invention

1) before enzymatic activation and
2) after enzymatic activation.

### DETAILED DESCRIPTION OF THE EXAMPLE EMBODIMENTS

Various example embodiments will now be described more fully with reference to the accompanying drawing in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawing and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figure.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figure. For example, functions/acts shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figure. It will be understood that the spatially relative terms are intended to encompass different orientations of the device in use or operation in addition to the orientation depicted in the figure. For example, if the device in the figure is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In one embodiment of the invention, a hyperpolarization site (polyhistidine or polytryptophan) is located at the N and/or C terminus of an MMP-7 substrate (proteoglycan, fibronectin, elastin, casein, or a short protease target sequence (RPLALWRS) which is surrounded by hydrophobic residues). The hydrophobic hyperpolarization site mediates aggregation of the MMP-7 substrates. In the aggregated state, the hyperpolarization site is inaccessible; only after proteolysis is this site exposed. In a further embodiment, protease sites of various proteases are combined (in particular MMP-7 and MMP-9, or MMP-7 and PSA, or MMP-9 and PSA) and therefore only the local and simultaneous activity of both proteases exposes the hyperpolarization sites. This enables an additionally higher specificity in the detection of highly aggressive tumors.

The construct according to an embodiment of the invention can be directed against any tumor-specific protease. Examples are MMP-1, MMP-2, MMP-3, MMP-7, MMP-9, MMP-10, MMP-11, MMP-12, MMP-14, MMP-15, MMP-16, MMP-17, PSA, uPA, and tPA.

The hyperpolarization site is, for example, the abovementioned heterocycle (heteroaromatic compound), for example the aromatic amino acids tryptophan or histidine.

The above-described construct is produced in a manner known per se according to the methods of classic protein synthesis.

In a first step for MR imaging, the above-described construct in the form of a prodrug is administered intravenously as a contrast agent. For this purpose, the construct is incorporated into a suitable formulation for imaging contrast agents. Such formulations are known in the field. The contrast agent is administered at dosages known per se. An exemplary dosage is 0.1 mmol of the contrast agent.

The contrast agent according to an embodiment of the invention accumulates in the target tissue (neovasculature in the tumor) owing to increased vascular permeability or is accumulated via a specific targeting mechanism. If the corresponding tumor-specific protease, for example MMP-7 or MMP-9, is expressed in the target tissue, the construct becomes fragmented and the hyperpolarization site becomes exposed. In a second step, the construct is contacted with a parahydrogen metal template or this is injected into the patient. Owing to the low MWT of the parahydrogen metal template, the bioavailability is high and said template reaches the target tissue within a few half-lives in order to hyperpolarize there the enzymatically activated construct. By means of established MR methods which are known in the prior art, the activity of the tumor-specific protease (e.g., MMP-7 activity) can be localized and a tumor can thus be diagnosed with specific accuracy. Examples of suitable imaging methods are MRI and sequence true FISP.

The method according to an embodiment of the invention is suitable for detecting any tumor type, for example neoplasias in the lung, breast, intestine, prostate, liver, neck, and head. The method is suitable for depicting neoplasias and/or precursors thereof.

Preferably, the method is carried out in patients who have been stratified beforehand as high-risk patients by way of laboratory tests. Suitable for this purpose is the detection of increased mRNA expression in fresh or fixed biopsies or tumor sections by means of array or PCR methods. Alternatively, in particular cases, the increased serum concentration of MMP-7 or MMP-9 protein can also be used. Corresponding methods for stratifying high-risk patients are known to a person skilled in the art.

Examples of possible uses in terms of combined in vitro and in vivo diagnostics which go beyond the scope of previous diagnostic options are presented below for prostate carcinoma by way of example, but without restricting the invention thereto. Rather, the method according to an embodiment of the invention represents an improvement in the diagnosis of other neoplastic diseases as well (lung, intestine, stomach, breast, ovary, neck and head, kidney, liver) and of different stages of the disease (TNM stages 1 to 4). An embodiment of the invention is suitable in particular for discriminating benign and malignant diseases and also for detecting at an early stage biochemically active micrometastases which have begun to affect the microenvironment in order to develop into macrometastases.

For a prostate carcinoma diagnosis that is improved according to an embodiment of the invention, particular preference is given to a contrast agent in which the protease cleavage sites for PSA, MMP-2, MMP-7, and/or MMP-9 are combined. A combination in particular which contains the PSA protease cleavage site is preferred since it results in high prostate specificity. The degree of severity of the disease can then be inferred from the activity at the combined MMP cleavage site. The prostate-specific antigen (PSA) is a serine protease which can degrade, inter alia, constituents of the extracellular matrix as well.

In the diagnosis of prostate carcinoma, increased PSA serum levels and further suspicions of a clinical nature, for example following digital rectal examination (DRE), conventional sonography, or transrectal ultrasound (TRUS), lead to multiple biopsies being taken in current practice. However, examination of the biopsies at the cellular level is often only insufficiently informative. Thus, the validity of the histological result is limited by the fact that the biopsy invariably comprises only parts of the prostate. For example, in the event of a positive biopsy, the spread of a prostate carcinoma can be conclusively assessed only after an operation. This leads in many cases to overtherapy owing to radical operation techniques, which also often lead to a significant and enduring reduction in the quality of life (incontinence, impotence). In contrast, if no pathological tissue can be detected (negative biopsy), this can mean that actually no carcinoma is present or else that it was not encountered, although, for example, the PSA level is increased or a lump is palpable. Thus, the biopsy cannot rule out a prostate carcinoma with absolute certainty. Therefore, despite the complications associated with prostate biopsy (bleedings, inflammations, allergy, acute ischuria, etc.), it is recommended to take up to 18 samples, depending on the prostate volume, as early as with the first biopsy. This increases the certainty of ruling out the carcinoma and also increases the detection rate. If results continue to be unclear, a repeated biopsy removal to a similar extent is required.

The method according to an embodiment of the invention for imaging a tumor tissue using the contrast agent according to an embodiment of the invention includes at least one of the following modifications and advantages compared with the corresponding methods from the prior art:
1) Measurement of additional serum markers, i.e., MMP-7 and MMP-9 in addition to the measurement of PSA. Increased MMP levels confirm the suspicion of a malignant disease and help in the selection of an adapted MMP- and/or PSA-specific contrast agent. Advantage: increased specificity of tumor detection due to combination with MMP markers.
2) Molecular analysis of mRNA expression of PSA, MMP1, MMP2, MMP7, MMP9, and MMP12 in tissue. Increased expression levels of, for example, MMP9 also indicate malignant transformation, even without the presence of tumor cells in the corresponding biopsy and help in the selection of an adapted MMP- and/or PSA-specific contrast agent. Advantages: biopsies lacking a tumor fraction also become diagnostically relevant owing to the tumor stroma reaction; the presence of specific MMP expression patterns differs depending on the degree of malignancy; selection of the downstream imaging method and of the most suitable contrast agent is made possible; the correlation of the MMP expression patterns with the position of the respective biopsies enables a comparative validation with downstream contrast agents directed against specific MMPs.
3) Stratified imaging by means of contrast agents according to an embodiment of the invention. Advantages: increased specificity by means of combined analysis of the measured serum protein levels, tissue expression levels, and activity levels recorded with an imaging method.
4) Adapted therapy which, in addition to conventional surgical, radiological, antihormonal, or chemotherapeutic therapy, also additionally comprises therapy directed against the matrix metalloproteases.
5) Monitoring after therapy has been effected, by way of repeated imaging with the corresponding contrast agent. Advantage: comparison before and after the therapy makes the effectiveness of the therapeutic measure quantifiable at an early stage, in particular in the case of tumor fractions remaining in the body (e.g., when treating bone metastases); an insufficient response to the selected therapy enables an early therapy modification.

The accompanying figure explains embodiments of the invention in detail.

The figure shows the construct according to an embodiment of the invention

1) before enzymatic activation and
2) after enzymatic activation.
R=hydrophobic substrate for hyperpolarization
L=linker
L₁/L₂=linker fragment after enzymatic cleavage
B=backbone (backbone structure)

The patent claims filed with the application are formulation proposals without prejudice for obtaining more extensive patent protection. The applicant reserves the right to claim even further combinations of features previously disclosed only in the description and/or drawing.

The example embodiment or each example embodiment should not be understood as a restriction, of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, non-transitory computer readable medium and non-transitory computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory storage medium or non-transitory computer readable medium, is adapted to store information and is adapted to interact with a data processing facility or computer device to execute the program of any of the above mentioned embodiments and/or to perform the method of any of the above mentioned embodiments.

The non-transitory computer readable medium or non-transitory storage medium may be a built-in medium installed inside a computer device main body or a removable non-transitory medium arranged so that it can be separated from the computer device main body. Examples of the built-in non-transitory medium include, but are not limited to, rewriteable non-volatile memories, such as ROMs and flash memories, and hard disks. Examples of the removable non-transitory medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A contrast agent for imaging methods, comprising a construct comprising
i) at least two copies of a substrate for at least one tumor-specific protease; and
ii) at least one linker including at least one recognition site for at least one tumor-specific protease,
wherein a hyperpolarization site is located at least one of an N and C terminus of the substrate, and wherein the at least one linker is configured such that hydrophobic ends of the substrate interact and form a central core by way of noncovalent interactions with the lipophilic residues.

2. The contrast agent as claimed in claim 1, wherein multiple linkers are bound to one backbone structure, and wherein the backbone structure is a physiologically compatible polymer.

3. The contrast agent as claimed in claim 1, wherein the tumor-specific protease is selected from the group consisting of MMP-1, MMP-2, MMP-3, MMP-7, MMP-9, MMP-10, MMP-11, MMP-12, MMP-14, MMP-15, MMP-16, MMP-17, uPA, tPA, and PSA.

4. The contrast agent as claimed in claim 1, wherein the at least one linker is a peptide.

5. The contrast agent as claimed in claim 1, wherein the at least one linker additionally carries space-filling groups for steric shielding of the central core.

6. The contrast agent as claimed in claim 1, wherein the hyperpolarization site is selected from the group consisting of polyhistidine and polytryptophan.

7. The contrast agent as claimed in claim 1, together with a parahydrogen metal template for use in an imaging method for diagnosing a tumor.

8. The contrast agent for the use as claimed in claim 7, wherein the imaging method is selected from the group consisting of MRI and sequence true FISP.

9. The contrast agent for the use as claimed in claim 7, wherein the tumor is selected from neoplasias of the lung, breast, intestine, prostate, liver, neck, and head.

10. A method for imaging a tumor tissue, comprising:
imaging the contrast agent, as claimed in claim 1 and accumulated in a tumor tissue, using a suitable imaging method after hyperpolarization by way of contact with a parahydrogen metal template.

11. The method as claimed in claim 10, wherein the imaging method is selected from the group consisting of MRI and sequence true FISP.

12. The method as claimed in claim 10, wherein the tumor is selected from neoplasias of the lung, breast, intestine, prostate, liver, neck, and head.

13. A method for imaging a tumor tissue in a patient, comprising:
a) administering a contrast agent as claimed in claim 1 to a patient;
b) administering a parahydrogen metal template to the patient; and
c) depicting a presence of a tumor using an imaging method.

14. The method as claimed in claim 13, wherein the imaging method is selected from the group consisting of MRI and sequence true FISP.

15. The method as claimed in claim 13, wherein the tumor tissue is selected from neoplasias of the lung, breast, intestine, prostate, liver, neck, and head.

16. The method as claimed in claim 13, wherein the patient has been stratified beforehand as a high-risk patient by way of a laboratory test.

17. The contrast agent as claimed in claim 2, wherein the tumor-specific protease is selected from the group consisting of MMP-1, MMP-2, MMP-3, MMP-7, MMP-9, MMP-10, MMP-11, MMP-12, MMP-14, MMP-15, MMP-16, MMP-17, uPA, tPA, and PSA.

18. The contrast agent as claimed in claim 4, wherein the peptide is Ala-Gly-Cys(Me)-His-Ala-Lys(Nma)-NH₂.

19. The contrast agent for the use as claimed in claim 8, wherein the tumor is selected from neoplasias of the lung, breast, intestine, prostate, liver, neck, and head.

20. The method as claimed in claim 11, wherein the tumor is selected from neoplasias of the lung, breast, intestine, prostate, liver, neck, and head.
